(19) United States
(12) Patent Application Publication
(10) Pub. No.: US 2016/0041101 A1
(43) Pub. Date: Feb. 11, 2016

(54) METHOD AND DEVICE FOR DETECTING AND IDENTIFYING NOT EASILY VOLATILIZED SUBSTANCES IN A GAS PHASE BY MEANS OF SURFACE-ENHANCED VIBRATION SPECTROSCOPY

(71) Applicants: Laser-Laboratorium Göttingen e.V., Goettingen (DE); AIRSENSE Analytics GmbH, Schwerin (DE)

(72) Inventors: Hainer Wackerbarth, Kassel (DE); Lars Gundrum, Goettingen (DE); Bert Ungethuem, Schwerin (DE); Wolf Muenchmeyer, Ehra-Lessien (DE); Andreas Walte, Schwerin (DE)

(21) Appl. No.: 14/886,227

(22) Filed: Oct. 19, 2015

Related U.S. Application Data
(63) Continuation of application No. PCT/EP2014/057805, filed on Apr. 16, 2014.

(30) Foreign Application Priority Data
Apr. 18, 2013 (DE) 10 2013 103 954.5

(51) Int. Cl. G01N 21/65; G01N 21/3504; G01N 33/22

(52) U.S. Cl. CPC G01N 21/658; G01N 33/227; G01N 21/3504; G01N 2021/258

(57) ABSTRACT

The invention relates to identifying not easily volatilized substances, in particular hazardous material, in a gas phase. A measurement cell and gas supply installations connected to the measurement cell are heated, and a plasmonic surface arranged in the measurement cell is temperature-controlled such that the plasmonic surface has a lower temperature than the measurement cell and the gas supply installations. The gas phase is guided through the gas supply installations into the measurement cell such that the gas phase reaches the plasmonic surface. Substances adsorbed out of the gas phase on the plasmonic surface are analyzed by an optical process. Surface-enhanced Raman spectroscopy or surface-enhanced infrared spectroscopy may be used. Selectivity can be increased by combining both methods. Selectivity can be additionally increased by using a gas detector, preferably an ion-mobility spectrometer. Thus the false alarm rate is reduced without a loss of time.

### CROSS REFERENCE TO RELATED APPLICATIONS

This application is a continuation to International Application PCT/EP2014/057805 filed on Apr. 16, 2014, entitled “Verfahren und Vorrichtung zur Detektion und Identifizierung von in einer Gasphase vorliegenden schwer flüchtigen Substanzen mittels oberflächenverstärkter Vibrationsspektroskopie” and claiming priority to German Patent Application No. DE 10 2013 103 954.5, filed on Apr. 18, 2013 and entitled “Verfahren und Vorrichtung zur Detektion und Identifizierung von Gefahrstoffen mit mindestens einem optischen System”.

### FIELD

The present invention relates to a method of identifying not easily volatilized substances present in a gas phase, particularly to a method of identifying hazardous material. The not easily volatilized substances may already be in the gas phase or they may first be transferred into the gas phase.

Such methods and related devices are employed for recognizing and detecting chemical substances or compounds, particularly explosives and/or noxious substances, like toxic industrial chemicals, warfare agents and drugs.

### BACKGROUND OF THE INVENTION

The detection of explosives and/or highly toxic chemical compounds requires measurement methods having detection limits in the ppt to ppb range (ppb=part per billion, 1 billion=1*10⁹), particularly if the substances, like plastic explosives, have a very low vapor pressure. Often spectrometers are used for recognizing and detecting these chemical compounds.

If a sufficient amount of the substance to be examined is available, methods like FTIR (Fourier Transform InfraRed Spectroscopy) or Raman spectroscopy may be used for identification. A drawback of FTIR is that the substance has to be touched and transferred to an analysis surface (ATR window). With some explosives, like for example peroxides, this may cause an explosion.

In Raman spectroscopy, the sample is irradiated with high intensity laser light and the reflected light is analyzed. Although the sample is not touched, it is a drawback of Raman spectroscopy that the intensity of the laser light may be sufficient to cause an explosion. Further, the Raman spectrum may be superimposed with fluorescence from traces of fluorescent compounds.

Many chemicals as well as many explosives may be detected via their gas phase as they have a sufficiently high vapor pressure.

Methods for analyzing small amounts of a plurality of substances are known as Surface Enhanced Raman Spectroscopy (SERS) and Surface Enhanced Infrared Absorption Spectroscopy (SEIRA). These two methods are variants of the so-called Surface Enhanced Vibrational Spectroscopy (SEVS).

In both known methods SERS and SEIRA, so-called plasmonic substrates or surfaces are used, which are substrates or surfaces having a surface structure supporting the excitation of localized plasmons. This results in an amplification of the Raman scattering in case of SERS and of the infrared absorption in case of SEIRA. By means of these plasmonic substrates or surfaces, a detection limit is reached which is by several orders magnitude lower than in standard Raman spectroscopy or infrared absorption spectroscopy. In the application of plasmonic surfaces, typically liquids or droplets of liquids are applied to the surface. The use of plasmonic surfaces for analysis of gaseous substances, however, is told to be complicated, as gaseous substances are not or only in small amounts and non-uniformly adsorbed on a substrate such that a reliable detection is hindered despite the high amplification. A known approach for solving this problem is to create an enlarged effective plasmonically active surface in that, for example, a plurality of capillaries with plasmonically active surfaces is used. According to another approach, the substrate is cooled. Here, it is a disadvantage that a cooled substrate results in a strong adsorption of moisture at the substrate which hinders the adsorption and thus the detection of the substance to be measured.

Further known methods for the analysis of a gas phase are Ion Mobility Spectrometry (IMS) which is also designated as plasma chromatography, and other spectrometric methods, such as mass spectrometry.

In IMS, in contrast to other spectrometric methods, like for example mass spectrometry, no vacuum pumps are needed for creating a vacuum. Thus, ion mobility spectrometers are small and low-cost as compared to mass spectrometers. A disadvantage of an ion mobility spectrometer as compared to a mass spectrometer is its lower resolution. Due to the lower resolution, the frequency of false alarms may be higher than in mass spectrometry.

A general overview over IMS and its application is, for example, found in: G. A. Eiceman and Z. Karpas “Ion Mobility Spectrometry” (2nd. Edition, CRC, Boca Raton, 2005).

Many hazardous substances, like for example RDX-based explosives, only have low vapor pressures so that such hazardous substances, if they are in a gas phase, tend to adsorb at the walls of a gas-guiding channel so that their transport into a measurement cell is hindered.

A thermo-electrically cooled SERS sensor system for detecting volatile organic compounds is known from U.S. Pat. No. 6,947,132 B1. The system has a desorber which includes an adsorbate for pre-concentrating a gas mixture. After the pre-concentration, the desorber is heated up to release the gas mixture into a measurement cell. In the measurement cell, an SERS structure is arranged which is cooled by a thermoelectric cooler. The gas mixture reaches the cooled SERS structure. The selection of the temperature of the cooler allows for condensing certain analytes at the SERS structure as different analytes condense at different temperatures. Temperatures of 15° C. at which benzene condensates, of 9° C. at which toluene condensates, and of −5° C. at which MTBE condensates are mentioned. The known system further comprises a laser and a spectrometer for carrying out an SERS method.

From published US patent application US 2007/0140900 A1 it is known to cool a nano-structured surface for an SERS measurement with a thermoelectric cooler down to a temperature in the range of 0° C. to 20° C. at which trace chemicals of interest are adsorbed on the surface.

From published US patent application US 2012/0133932 A1 it is known to use a thermoelectric element at one time for cooling an SERS substrate to enhance the condensation and adsorption of a selected analyte, and at another time for heating the SERS substrate up to refresh its surface for a following analysis.

U.S. Pat. No. 6,610,977 B2 describes a security system in which an ion mobility spectrometry (IMS) sensor is combined with an SERS sensor as a second sensor. Such a combination is also disclosed in published US patent application US 2009/0238723 A1. The SERS sensor downstream of the IMS sensor records spectra of the ions generated in the IMS sensor instead of spectra of the starting substances.

There still is a need of a method and a device for identifying hazardous substances which are present in a gas phase or which may be transferred into the gas phase at low concentrations and at a high reliability.

### SUMMARY OF THE INVENTION

The present invention relates to a method of identifying of not easily volatilized substances present in a gas phase. The method comprises heating up a measurement cell and gas supply installations connected to the measurement cell, and adjusting a temperature of a plasmonic surface located in the measurement cell so that the plasmonic surface has a lower temperature than the measurement cell and the gas supply installations. The method further comprises supplying the gas phase through the gas supplying installations into the measurement cell so that the gas phase gets to the plasmonic surface, and applying an SEVS method including the irradiation of the plasmonic surface with electromagnetic radiation for identifying substances adsorbed on the plasmonic surface out of the gas phase.

The present invention also relates to a device for identifying not easily volatilized substances present in a gas phase. The device comprises a measurement cell, a plasmonic surface located in the measurement cell, a gas supply installation connected to the measurement cell, and at least one connector for a radiation source configured to irradiate the plasmonic surface with electromagnetic irradiation and for an optical detection device configured to apply an SEVS method. The device further comprises a heating device configured to heat the measurement cell and the gas supply installations connected thereto, a temperature adjusting device configured to adjust the temperature of the plasmonic surface in such a way that the plasmonic surface has a lower temperature than the heated measurement cell and gas supply installations, and gas-guiding installations configured to guide the gas phase through the gas supply installations into the measurement cell such that the gas phase gets to the plasmonic surface.

Other features and advantages of the present invention will become apparent to one with skill in the art upon examination of the following drawings and the detailed description. It is intended that all such additional features and advantages be included herein within the scope of the present invention, as defined by the claims.

### SHORT DESCRIPTION OF THE DRAWINGS

The invention can be better understood with reference to the following drawings. The components in the drawings are not necessarily to scale, emphasis instead being placed upon clearly illustrating the principles of the present invention. In the drawings, like reference numerals designate corresponding parts throughout the several views.

FIG. 1 shows the basic construction of one embodiment of the device according to the present invention.

FIG. 2 is a longitudinal section through a measurement cell and a gas-guiding system connected to the measurement cell of a further embodiment of the device according to the present invention.

FIG. 3 is a front view of the measurement cell and the gas-guiding system of the embodiment of the device according to the present invention according to FIG. 2; and

FIG. 4 is a block diagram of an embodiment of the method according to the present invention.

### DETAILED DESCRIPTION

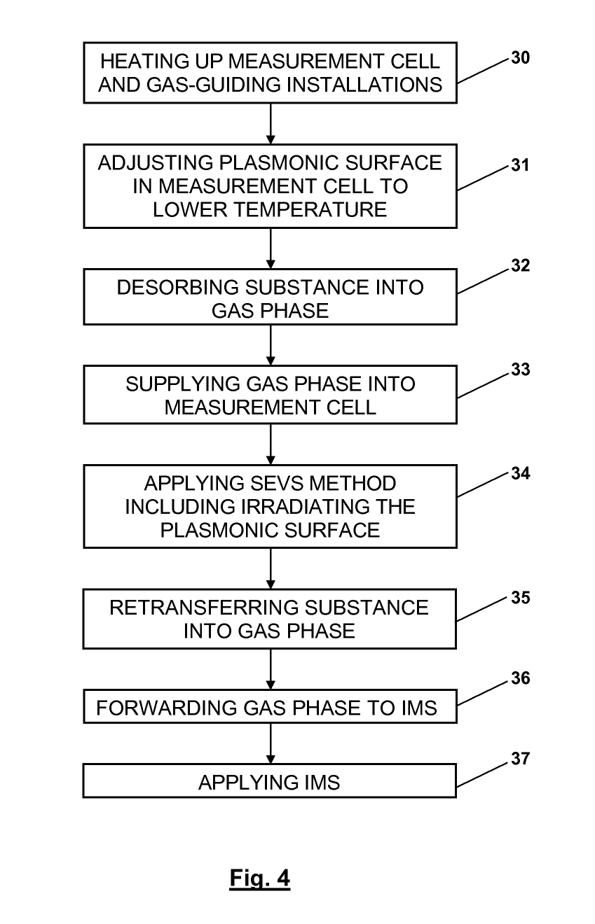

An embodiment of the method according to the present invention comprises the steps of:
heating up a measurement cell and gas-guiding installations connected to the measurement cell, see step 30 in FIG. 4,
adjusting a temperature of a plasmonic surface arranged in the measurement cell such that the plasmonic surface has a lower temperature than the measurement cell and the gas supplying installations, see step 31 in FIG. 4,
supplying a gas phase into the measurement cell through the gas supplying installations such that the gas phase reaches the plasmonic surface at the
applying an SEVS method including irradiation of the plasmonic surface with electromagnetic radiation for detecting hazardous substances included in the gas phase, see step 34 in FIG. 4.

Adjusting or controlling the temperature of the plasmonic surface may require active cooling of the plasmonic surface. Hazardous substances included in the gas phase are detected based on those parts of the substance adsorbed out of the gas phase on the cooler plasmonic surface. The step of heating, besides heating the measurement cell and the gas supplying installation, may include heating an entire gas-guiding system connected to the measurement cell. The step of heating inhibits the adsorption of substances at the inner surfaces of the measurement cell and of the gas-guiding system. In this way, a contamination of the inner walls of the measurement cell and of the gas-guiding system by means of adsorbed substances is prevented. This particularly inhibits that such substances are carried over into a following measurement. Further, the detection limit of the method according to the invention is optimized, as the substances of interest may only adsorb on the plasmonic surface.

In case of SERS being the analysis method, preferably laser light is used as the electromagnetic radiation. Both in SERS and SEIRA also wavelengths outside the visible spectral range may be used, as they are typically used for SERS and SEIRA. These wavelengths and suitable sources of electromagnetic radiation in case of SERS and SEIRA are known to those skilled in the art.

The reliability of the measurement may be increased in that the method according to the present invention may be combined with an independent further measurement method. For example, a combination of SERS and SEIRA analytics may be applied in the measurement cell.

Further, it is advantageous to apply a combination of the SEVS analytics with a non-optical gas detection method.

Here, it is generally advantageous, if the gas phase is first forwarded into the SEVS measurement cell and only in a next step forwarded into a further analysis module. This advantage results from the fact that the substances to be detected are generally not chemically or physically altered in the SEVS measurement cell.

As a downstream analysis method, IMS may advantageously be used. Particularly in the analysis of substance mixtures, it may occur that a separation of the spectra belonging to the individual substances is not possible at a sufficient reliability and that it is unclear by which number of substances included in a sample the combined spectrum is generated. IMS, however, reliably separates the mixtures in separate fractions so that the number of substances included in the sample may be concluded from the IMS data at a high reliability. This knowledge may then advantageously be used in the chemometric analysis of the SEVS data.

Particularly in IMS it is advantageous to direct the gas phase first into the SEVS measurement cell, as in IMS an ionization of the sample substance takes place so that, with an inversed sequence, i.e. with first directing the gas phase into the IMS device and only afterwards into the SEVS cell, only ions of the starting substance and probably ionized derivates of the starting substance may be detected in the SEVS cell.

The set of measurement values produced in the method according to the present invention is supplied to an evaluation unit. Here, the SEVS spectra are preferably analyzed by methods of chemometry. If the SEVS measurement method is combined with a further measurement method, the data are evaluated together, i.e. in evaluating the measurement results of one method, the knowledge derived from the evaluation of the respective other method(s) is considered, or the data are first fused, and then the fused data are chemometrically analyzed as a whole, or a mutual analysis of the measurement results and a data fusion of data from several measurement methods are combined in the evaluation.

In the method according to the present invention, the plasmonic surface may successively be heated up, and analysis data of the gas detector as well as, optionally, also of the SEVS method may be related to the respective temperature of the plasmonic surface. In this way, the analysis data of the gas detector and also of the SEVS method may be related to individual substances which differ by their vapor pressure or their boiling point.

The method according to the present invention, allows for detecting substances having vapor pressures at 30° C. of not more than 6.46 Pa, which is the vapor pressure of triacentontriperoxide (TATP).

In the laboratory of the assignee Laser-Laboratorium Göttingen, measurements with TATP have been carried out. For this purpose, a container including TATP in a gas phase was heated up to 45° C. In this way, air was saturated with TATP. This air was supplied to a measurement cell. In the measurement cell, an SERS substrate comprising the plasmonic surface was adjusted to a fixed temperature. In a series of measurements the temperature was adjusted to 25° C., 20° C., 15° C., 10° C. and 5° C.

Already at 25° C., the measurement results clearly showed SERS intensity signals. At a temperature of 5° C., the signal height was increased by a factor about 2.5. The signal quality, determined as the edge steepness and the full width of half maximum of the Raman bands, as an indicator of the signal-to-noise ratio, was even increased by a factor of about 2.8.

From further experience, the inventors knew that a further cooling of the SERS substrate down to the refrigeration point or even below results in an adsorption of moisture at the SERS substrate.

From common experiments of both assignees it is known that, even for not easily volatilized substances, like for example TNT and/or RDX-based explosives, an effective suppression of the adsorption in the gas-guiding system is achieved, if the gas-guiding system and the measurement cell are heated up to temperatures of about 160° C. or above. Here, it is to be noted that the preferably used temperature may be dependent on the actual measurement task. Thus, for hazardous substances, like TATP and EDN (ethylenglycoldinitrate=nitroglycol) having comparatively high vapor pressures as compared to the group of substances to be detected according to the present invention, a lower temperature may be sufficient, whereas particularly for RDX-based explosives and for PETN (other names: nitropenta, pentrite, pentaerythrityltetranitrate) comparatively higher temperatures are needed. With all substances, an increased temperature reduces the tendency of adsorption in the gas-guiding system. Temperature limits may result from the ignition temperature and/or the decomposition temperature of the substances to be detected. Typically, the gas-guiding system and the measurement cell are heated to temperatures in a range from 150° C. to 200° C.

By experiments of the inventors it has further been proven that reliably evaluatable TNT spectra are obtained by cooling an SERS surface to 80° C. As TNT has a lower vapor pressure than RDX, it may be assumed that cooling to 80° C. is also sufficient for RDX detection.

That cooling the plasmonic surface to such comparatively high temperatures above room temperature is sufficient, turns out to be advantageous as it limits the amount of heat to be removed at the comparatively high temperature to which the gas-guiding system and the measurement cell are to be heated.

Preferably, however, the plasmonic surface is cooled down further to, for example, 5° C.

So far as it cannot be excluded that the gas phase includes moisture, cooling should only take place down to a temperature which is still above the freezing point of water. The plasmonic surface may also be cooled step by step combined with an analysis of the adsorbed substances in each step.

If a liquid or solid substance is to be examined to see whether it includes an hazardous substance which only gets into a surrounding gas phase to a small extent, because the hazardous substance, for example, only has a very small vapor pressure and/or because the substance to be examined only includes little hazardous substance or because the time over which the hazardous substance may be enriched in a gas volume is limited, a thermal desorption method or a corresponding device may be used in the present invention.

From experiments at the assignee Airsense Analytics GmbH is it known that temperatures of 200° C. and above are preferably used for the thermal desorption of RDX-based hazardous substances. The temperature at which the desorption takes place may also be increased step by step while analyzing the substances adsorbed on the plasmonic surface in each of the steps.

In the application of the method according to the present invention for identifying gases it is an advantage that due to taking the sample out of the gas phase, the substances are enriched at a suitable surface without touching them. This surface may, for example, be cooled down by means of a Peltier element or a Stirling cooler.

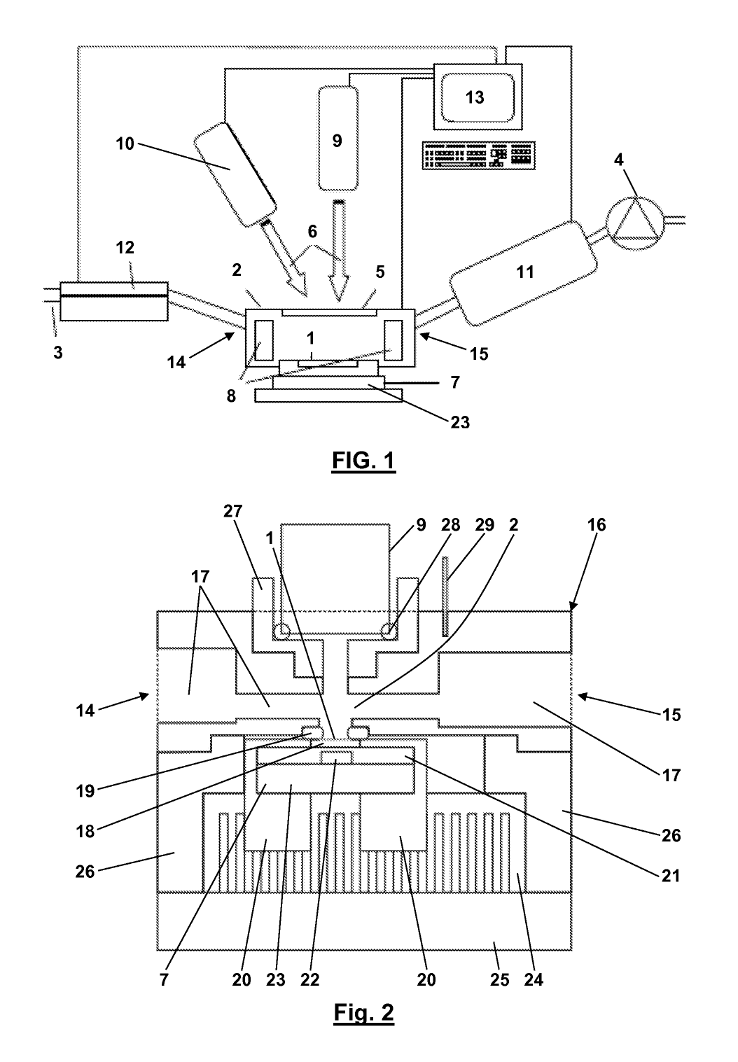

Now referring in greater detail to the drawings, a sample, if gaseous, is directly sucked into a measurement cell 2 of the device according to FIG. 1. Alternatively, it is placed in a thermal desorber 12 or thermodesorber by means of an adsorption fleece. In the thermal desorber 12, the substances included in the sample are heated up and desorbed, see step 32 of FIG. 4. Desorption temperatures of higher than 200° C. may be used in case of RDX-based explosives. Form the thermal desorber 12 the gas phase including the desorbed sample in air which is sucked in through an inlet 3 is forwarded to a plasmonic surface located within the measurement cell 2, see step 32 of FIG. 4. Sucking the gas phase in is controlled by means of a gas pump 4.

The measurement cell 2 and gas supplying installations 14 and gas removing installations 15 connected to the measurement cell are heated by heating elements 8 only schematically depicted in FIG. 1. For the air-based transport of not easily volatilized explosives like RDX, heating takes place up to temperatures of about 160° C. and above to avoid the precipitation of the molecules of the explosives at the inner walls of the measurement cell 2 and of the gas supplying installations and gas removing installations 15. To minimize the adsorption of explosives and like at any inner walls, small volumina of the measurement cell 2 and of the gas supplying installations and gas removing installations 15 are preferred and all their parts are heatable.

The plasmonic surface 1 is an especially structured surface of a plasmonic substrate interacting with laser light 6 in such a way that a signal excited by means of the laser light 6 is amplified. The plasmonic substrate is cooled by a peltier element 23 provided as a cooling element 7. Thus, a difference in temperature is generated so that molecules which get into contact with the plasmonic surface 1 are deposited. For example, the plasmonic surface 1 has a by 80 K lower temperature than the measurement cell 2. The molecules deposited on the plasmonic substrate are detected with the aid of the laser light 6 by means of an SEVS method, for example by SERS or SEIRA spectroscopy, or both by SERS and SEIRA spectroscopy. For this purpose, a Raman spectrometer 9 and an IR spectrometer 10 are provided. The laser light 6 is radiated from the respective spectrometer 9 or 10 through a window 5 into the measurement cell 2.

In the device according to the FIG. 1, a gas detector 11 for carrying out a further analysis method is connected downstream of the measurement cell 2. For this further analysis method, the molecules are desorbed from the plasmonic surface 1 by heating up the plasmonic surface, see step 35 of FIG. 4. Heating up of the plasmonic surface may take place by switching off the cooling element 7 and/or by using additional heating elements for the plasmonic substrate not depicted here.

During the entire time the gas pump 4 sucks in. If the gas detector 11 is connected, an external gas pump may be avoided, as a rule, as gas detectors have internal gas pumps. With an IMS as the gas detector 11, the molecules which are not adsorbed on the plasmonic substrate may directly be ionized and analyzed. The gas detector 11 also measures during heating the plasmonic substrate As a result, two gas packages with different loads reach the gas detector 11 during one measurement process, one without and one including the desorbed substances. In FIG. 1, the combination SEVS (SERS and/or SEIRA)-IMS is depicted.

The thermal desorber 12 may provide a starting signal causing the SERS spectrometer being the combination of the Raman spectrometer 9 and the plasmonic surface 1, the SEIRA spectrometer being the combination of the IR spectrometer 10 and the plasmonic surface 1, and the gas detector 11, for example being an IMS spectrometer, to start to detect. Data from the SERS spectrometer, the SEIRA spectrometer and the IMS spectrometer are read out and forwarded to a computer 13 on which an analysis software is running. Here, the data are processed and analyzed by mathematical-chemometrical methods. All data are analyzed and weighted. Prior to heating the plasmonic surface land during heating the plasmonic surface 1, the signals of the IMS, SERS or SEIRA spectrometers are recorded. Fusion of the data takes place in the computer 13. Here, the measurement results during enrichment and during heating up are compared. The comparison helps in the interpretation of the data, as, for example, easily volatilized compounds are not enriched on the substrate whereas not easily volatilized compounds may be enriched on the substrate. Additionally, the results of the individual optical detectors shall be compared with the results of the gas detector 11. The quality of the results of the spectra as compared to data bank spectra of each individual spectroscope or spectrometer is to be determined. Afterwards, the results of the individual detectors are weighted and summed up to come to an overall result. Depending on the identification, criteria for exclusion are to be defined. If, for example, a substance may only be identified as “A or B” by means of SERS, but the IMS spectrometer responds to the substances A and B differently, an exclusion criterion for the result “substance A” or “substance B” may be defined by means of additional weighting factors. These criteria are to be determined experimentally.

The result of the evaluation is output depending on the situation. With inspections in airports, for example, an alarm signal may be triggered in case of detection of explosives. For use by police or fire brigades, the name of the substance or its composition may be indicated.

The device according to FIG. 1 is constructed in modules, i.e. the thermal desorber 12, the spectrometers 9 and 10, the measurement cell 2 and the gas detector 11 are individual modules. The Raman spectrometer 9 may, for example, be operated in combination with the desorber 12 only. The thermal desorber 12 may be omitted, if the device is to be integrated in gates or air conditioners. The modularity of the device allows for a high flexibility in adapting the device to varying conditions.

An identification of a not easily volatilized substance present in a gas phase, particularly of an hazardous substance, may be executed by means of the device according to FIG. 1 as follows: In the thermal desorber 12 an analyte, like for example an explosive, is desorbed at a temperature of equal to or above 200° C. into a gas phase otherwise consisting of air, see step 32 of FIG. 4. The gas phase is introduced into the measurement cell 2, see step 33 of FIG. 4. Here, the gas supplying installations 14 and the measurement cell are kept at temperatures between 150° C. and 200° C., for example, which avoid condensing of the analyte out of the air, see step 30 of FIG. 4. In the measurement cell, the analyte precipitates at the plasmonic surface 1 due to the cooling of the plasmonic substrate, see step 31 of FIG. 4. With many explosives, cooling the plasmonic substrate to about 50° C. is sufficient for this purpose. Then, spectra of the analyte are recorded with the spectrometers 9 and 10, see step 34 of FIG. 4. After recording the spectra, the plasmonic substrate is heated up to desorb the analyte, see step 35 of FIG. 4. The desorbed analyte is sucked in by the gas detector 11, see step 36 of FIG. 4, and detected there, see step 37 of FIG. 4. During the entire time (about 30 s) needed for this measurement, the gas detector 11 records data (one spectrum per second). In this way, besides the data from the gas detector, for example an IMS spectrometer, meta data of the dynamics of the release of the analyte in the thermal desorber and from the plasmonic surface 1 as well as of its transport are generated. By means of these meta data, inter alia the temporal course of the spectra recorded by the IMS spectrometer are described, and the temperatures of the different parts are recorded as a function of time. Thus, the kinetics of the analysis process, particularly of the adsorption on and the desorption from the plasmonic surface 1, are described. The kinetics of these processes are specific for different substances as they are inter alia determined by the vapor pressure and the adsorption energy of the analyte on the surfaces of the desorber 12 and the plasmonic substrate. In short, by means of the temporal changes of the signals in the spectra of the gas detector 11 in combination with the recorded temperatures, additional information is generated which may be used for substance identification.

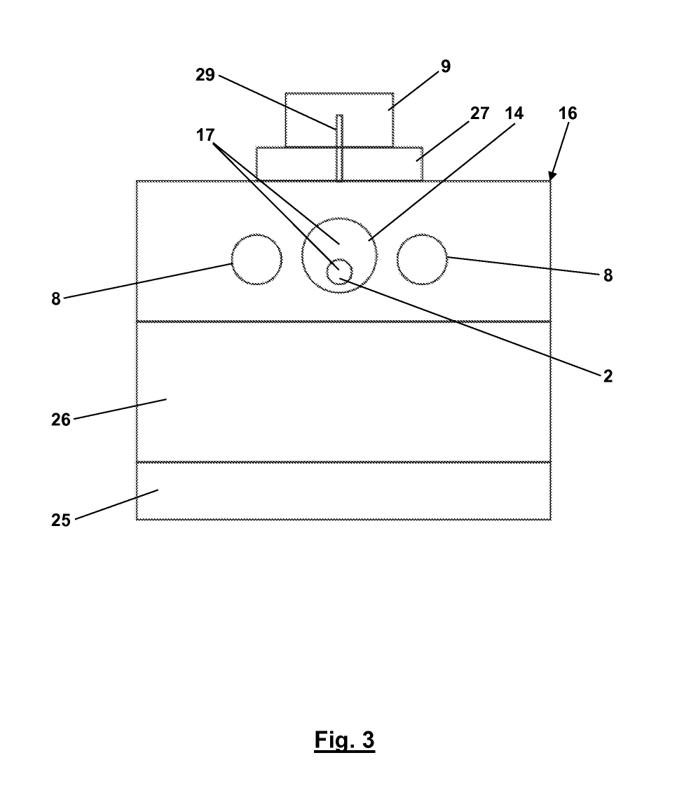

The measurement cell 2 depicted in FIGS. 2 and 3 together with attached gas supply installations 14 and gas removing installations 15 comprises a shaped body 16 made of stainless steel. A gas channel 17 runs through the shaped body 16. In the area of the measurement cell 2, the diameter of the gas channel 17 is a few millimeters only to keep the volume of the measurement cell 2 small. Parallel to the gas channel 17, heating elements 8 are provided in the shaped body 16 by which the measurement cell 2 and the adjoining gas supply installations 14 and gas removing installations 15 may be heated up. The temperature of the shaped body 16 is surveyed by means of a temperature sensor 26. The plasmonic substrate 18 with the plasmonic surface 1 adjoins the gas channel 17 in the measurement cell 2. Here, the plasmonic substrate 18 is thermally decoupled from the shaped body 16 by means of an O-ring 19, and it is held by substrate holders 20 made of thermally insulating polyetherketone. The substrate holders 20 also hold a substrate carrier 21 adjoining the substrate 18 at its back and comprising a channel for a temperature sensor 22 and a Peltier element 23 at the backside of the substrate carrier 21 serving as a cooling element 7. At the backside of the Peltier element 23 a cooling fin body 24 made of aluminum is arranged whose cooling efficiency is increased by means of a fan 25. The cooling fin body and the substrate holder 20 are laterally surrounded by thermal insulators 26 which are also made of polyetherketone. The Raman spectrometer 9 is facing the plasmonic surface 1 across the gas channel 17, wherein further thermal insulators 27 made of polyetherketone and an O-ring 28 are provided between the shaped body 16 and the Raman spectrometer 9.

Between the Raman spectrometer 9 and the gas channel 17, a window for delimiting the measurement cell 2 may be provided which is not depicted here and which, if provided, is also heated like the adjoining shaped body 16 to avoid an absorption due to condensation by the substances to be identified.

Many variations and modifications may be made to the preferred embodiments of the invention without departing substantially from the spirit and principles of the invention. All such modifications and variations are intended to be included herein within the scope of the present invention, as defined by the following claims.

We claim:

1. A method of identifying of not easily volatilized substances present in a gas phase, the method comprising:
heating up a measurement cell and gas supply installations connected to the measurement cell,
adjusting a temperature of a plasmonic surface located in the measurement cell so that the plasmonic surface has a lower temperature than the measurement cell and the gas supply installations,
supplying the gas phase through the gas supplying installations into the measurement cell so that the gas phase gets to the plasmonic surface,
applying an SEVS method including the irradiation of the plasmonic surface with electromagnetic radiation for identifying substances adsorbed on the plasmonic surface out of the gas phase.

2. The method of claim 1, wherein the measurement cell and the gas supply installations are heated up to a temperature of at least 160° C.

3. The method of claim 1, wherein the plasmonic surface is cooled down to a temperature of 80° C. or below.

4. The method of claim 1, wherein the substances adsorbed on the plasmonic surface are analyzed optically by means of at least one of surface-enhanced Raman spectroscopy (SERS) or surface-enhanced infrared spectroscopy (SEIRA).

5. The method of claim 1, wherein substances which are guided in the gas phase over the plasmonic surface and which are not adsorbed on the plasmonic surface are forwarded within the gas phase to a gas detector and analyzed by means of the gas detector.

6. The method of claim 1, wherein substances which have been adsorbed on the plasmonic surface are retransferred into the gas phase by heating up the plasmonic surface after application of the SEVS method, and wherein the retransferred substances are forwarded to a gas detector and analyzed by means of the gas detector.

7. The method of claim 6, wherein the plasmonic surface is heated up step by step and wherein analyses of at least one of the gas detector and the SEVS method are related to the respective temperature of the plasmonic surface.

8. The method of claim 6, wherein the gas detector is an ion mobility spectrometer.

9. The method of claim 1, wherein the plasmonic surface is cleaned after the identification of each not easily volatilized substance.

10. The method of claim 9, wherein the plasmonic surface, for cleaning, is heated up to a cleaning temperature for a defined period of time.

11. The method of claim 9, wherein the plasmonic surface, for cleaning, is subjected to at least one chemical cleaning compound for a defined period of time.

12. The method of claim 11, wherein the plasmonic surface is subjected to ozone as the at least one chemical cleaning compound.

13. The method of claim 11, wherein the plasmonic surface, after being subjected to the at least one chemical cleaning compound, is rinsed with clean air.

14. The method of claim 1, wherein the non-volatilized substances are transferred into the gas phase by means of a thermal desorber prior to supplying the gas phase through the gas supplying installations into the measurement cell.

15. The method of claim 14, wherein the not easily volatilized substances are heated up in the thermal desorber up to a desorption temperature of at least 200° C.

16. The method of claim 1, wherein results of the SEVS method during adsorption at the plasmonic surface are compared to results of the SEVS method during heating up the plasmonic surface.

17. The method of claim 5, wherein the different analyses are individually evaluated to provide individual results and wherein the individual results are weighted prior to combining them to a total result.

18. A device for identifying not easily volatilized substances present in a gas phase, the device comprising
a measurement cell,
a plasmonic surface located in the measurement cell,
a gas supply installation connected to the measurement cell,
at least one connector for a radiation source configured to irradiate the plasmonic surface with electromagnetic irradiation and for an optical detection device configured to apply an SEVS method,
a heating device configured to heat the measurement cell and the gas supply installations connected thereto,
a temperature adjusting device configured to adjust the temperature of the plasmonic surface in such a way that the plasmonic surface has a lower temperature than the heated measurement cell and gas supply installations, and
gas-guiding installations configured to guide the gas phase through the gas supply installations into the measurement cell such that the gas phase gets to the plasmonic surface.

19. The device of claim 18, wherein a gas-guiding system including the gas supply installations comprises a gas pump.

20. The device of claim 18, wherein the measurement cell comprises a window permeable for the electromagnetic irradiation.

21. The device of claim 18, wherein the temperature adjusting device comprises a cooling element.

22. The device of claim 18, wherein the radiation source configured to irradiate the plasmonic surface with electromagnetic radiation and the optical detection device for the application of an SEVS method are connected to the at least one connector.

23. The device of claim 22, wherein the optical detection device comprises at least one of a Raman spectrometer configured to generate SERS spectra using the plasmonic surface and an infrared spectrometer configured to generate SEIRA spectra using the plasmonic surface.

24. The device of claim 23, wherein the infrared spectrometer is a Fourier-transform-infrared spectrometer.

25. The device of claim 18, wherein a gas detector is connected to an output of the measurement cell.

26. The device of claim 25, wherein the gas detector is an ion mobility spectrometer.

27. The device of claim 18, wherein the gas supply device is connected to a thermal desorber configured to thermally desorb samples.
